SEQUENCE LISTING (4 sequences)

SEQ ID NO: 1 - DNA, 666 bp, Artificial Sequence
  TAX2 gene
gcccatttcccaggatttggacagagcctcctatatggataccccgtctacgtgtttggcgattgtgtacaggccgattggtgtcccgtctcaggtggtctatgttccacccgcctacatcgacatgccctcctggccacctgtccagagcaccaactcacctgggaccccatcgatggacgcgttgtcagctctcctctccaataccttatccctcgcctcccctccttccccacccagagaacctcaaggaccctcaaggtccttacccctcccaccactcctgtctcccccaaggttccacctgccttctttcaatcaatgcgaaagcacaccccctaccgaaatggatgcctggaaccaaccctcggggatcagctcccctccctcgccttccccgaacctggcctccgtccccaaaacatctacaccacctggggaaaaaccgtagtatgcctatacctataccagctttccccacccatgacatggccacttataccccatgtcatattctgccaccccagacaattaggagccttcctcaccaaggtgcctctaaaacgattagaagaacttctatacaaaatgttcctacacacagggacagtcatagtcctcccggaggacgacctacccaccacaatgttccaacccgtgagggct

SEQ ID NO: 2 - DNA, 1818 bp, Artificial Sequence
  41BBL-MICA fusion gene
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaagccacgaacttctctctgttaaagcaagcaggagatgttgaagaaaaccccgggcctatggggctgggcccggtcttcctgcttctggctggcatcttcccttttgcacctccgggagctgctgctgagccccacagtcttcgttataacctcacggtgctgtcctgggatggatctgtgcagtcagggtttcttgctgaggtacatctggatggtcagcccttcctgcgctatgacaggcagaaatgcagggcaaagccccagggacagtgggcagaagatgtcctgggaaataagacatgggacagagagaccagggacttgacagggaacggaaaggacctcaggatgaccctggctcatatcaaggaccagaaagaaggcttgcattccctccaggagattagggtctgtgagatccatgaagacaacagcaccaggagctcccagcatttctactacgatggggagctcttcctctcccaaaacctggagactgaggaatggacagtgccccagtcctccagagctcagaccttggccatgaacgtcaggaatttcttgaaggaagatgccatgaagaccaagacacactatcacgctatgcatgcagactgcctgcaggaactacggcgatatctagaatccggcgtagtcctgaggagaacagtgccccccatggtgaatgtcacccgcagcgaggcctcagagggcaacatcaccgtgacatgcagggcttccagcttctatccccggaatatcatactgacctggcgtcaggatggggtatctttgagccacgacacccagcagtggggggatgtcctgcctgatgggaatggaacctaccagacctgggtggccaccaggatttgccgaggagaggagcagaggttcacctgctacatggaacacagcgggaatcacagcactcaccctgtgccctctgggaaagtgctggtgcttcagagtcattggcagacattccatgtttctgctgttgctgctggctgctgctatttttgttattattattttctatgtccgttgttgtaa

SEQ ID NO: 3 - DNA, 762 bp, Artificial Sequence
  41BBL gene
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 4 - DNA, 999 bp, Artificial Sequence
  MICA gene
atggggctgggcccggtcttcctgcttctggctggcatcttcccttttgcacctccgggagctgctgctgagccccacagtcttcgttataacctcacggtgctgtcctgggatggatctgtgcagtcagggtttcttgctgaggtacatctggatggtcagcccttcctgcgctatgacaggcagaaatgcagggcaaagccccagggacagtgggcagaagatgtcctgggaaataagacatgggacagagagaccagggacttgacagggaacggaaaggacctcaggatgaccctggctcatatcaaggaccagaaagaaggcttgcattccctccaggagattagggtctgtgagatccatgaagacaacagcaccaggagctcccagcatttctactacgatggggagctcttcctctcccaaaacctggagactgaggaatggacagtgccccagtcctccagagctcagaccttggccatgaacgtcaggaatttcttgaaggaagatgccatgaagaccaagacacactatcacgctatgcatgcagactgcctgcaggaactacggcgatatctagaatccggcgtagtcctgaggagaacagtgccccccatggtgaatgtcacccgcagcgaggcctcagagggcaacatcaccgtgacatgcagggcttccagcttctatccccggaatatcatactgacctggcgtcaggatggggtatctttgagccacgacacccagcagtggggggatgtcctgcctgatgggaatggaacctaccagacctgggtggccaccaggatttgccgaggagaggagcagaggttcacctgctacatggaacacagcgggaatcacagcactcaccctgtgccctctgggaaagtgctggtgcttcagagtcattggcagacattccatgtttctgctgttgctgctggctgctgctatttttgttattattattttctatgtccgttgttgtaa

(19) United States
(12) Patent Application Publication
(10) Pub. No.: US 2023/0065434 A1
(43) Pub. Date: Mar. 2, 2023

(54) PREPARATION METHOD OF TROPHOBLASTS WITH LIMITED GENERATIONS, CULTURE METHOD OF SNK CELLS AND METHOD FOR TREATING TUMOR

(71) Applicant: BEIJING DCTY BIOTECH CO., LTD., Beijing (CN)

(72) Inventors: Shunchang JIAO, Beijing (CN); Rong ZHANG, Beijing (CN); Zishan ZHOU, Beijing (CN)

(21) Appl. No.: 17/758,190

(22) PCT Filed: Dec. 18, 2020
(86) PCT No.: PCT/CN2020/137551

§ 371 Date: Jun. 29, 2022

(30) Foreign Application Priority Data
Dec. 30, 2019 (CN) 201911401406.X

(51) Int. Cl. C12N 15/86; C12N 5/0783; A61K 35/17; A61P 35/00

(52) U.S. Cl. CPC C12N 15/86; C12N 5/0646; A61K 35/17; A61P 35/00; C12N 2740/15043; C12N 2506/115; C12N 2501/2302

(57) ABSTRACT

Provided are a preparation method of trophoblasts with limited generations, a culture method of SNK cells and a method for treating tumor. The preparation method of trophoblasts includes the following steps: ligating a TAX2 gene to a lentiviral expression vector, followed by transferring into competent cells to obtain a lentivirus containing the TAX2 gene; infecting PBMCs with the lentivirus containing the TAX2 gene and culturing, and collecting CD3-cells; ligating a 41BBL-MICA fusion gene to the lentiviral expression vector, followed by transferring into the competent cells to obtain a lentivirus containing the 41BBL-MICA fusion gene; and mixing the CD3-cells with the lentivirus containing the 41BBL-MICA fusion gene and culturing to obtain the trophoblasts with limited generations.

### CROSS REFERENCE TO RELATED APPLICATION

The present disclosure claims priority of Chinese Patent Application No. 201911401406.X filed to the China National Intellectual Property Administration (CNIPA) on Dec. 30, 2019 and entitled “PREPARATION METHOD OF TROPHOBLASTS WITH LIMITED GENERATIONS, CULTURE METHOD OF SNK CELLS AND METHOD FOR TREATING TUMOR”, which is incorporated by reference herein in its entirety.

### TECHNICAL FIELD

The present disclosure belongs to the technical fields of medicine, immunology, cell biology and molecular biology, and in particular relates to a preparation method of trophoblasts with limited generations, a culture method of SNK cells and a method for treating a tumor.

### BACKGROUND ART

Common culture methods of natural killer (NK) cells are as follows: 1. isolating NK cells from autologous peripheral blood mononuclear cells (PBMCs), and culturing; however, this method has the disadvantages of small expansion number, low CD56+CD16+ ratio and low NK activity. 2. Induced culturing of NK cells from allogeneic stem cell source; however, this method has the disadvantages of allogeneic source risk, the low CD56+CD16+ ratio and the low NK activity. 3. Using K562 as trophoblasts; since the K562 is a tumor cell line, there is a certain risk in use and previous irradiation is needed before use, which increases the difficulty of use; moreover, the CD56+CD16+ ratio is low, and the NK cells only have a certain killing activity on B cells or lymphoma and a low killing activity on solid tumors.

### SUMMARY

Based on the above reasons, the present disclosure provides a preparation method of trophoblasts with limited generations, a culture method of SNK cells and a method for treating a tumor. The trophoblasts cultured by the culture method provided by the present disclosure are limited generation expansion cells and have no tumorigenic risk in vivo, and can stimulate expansion of CD56+CD16+NK cells in PBMCs (peripheral blood mononuclear cells) from different sources; the obtained NK cells have a relatively strong killing effect on various solid tumors.

The present disclosure is achieved by the following technical schemes.

The present disclosure provides a preparation method of trophoblasts with limited generations, including the following steps:

1) ligating a TAX2 gene to a lentiviral expression vector, followed by transferring into competent cells to obtain a lentivirus containing the TAX2 gene;

2) infecting peripheral blood mononuclear cells (PBMCs) with the lentivirus containing the TAX2 gene obtained in step 1) and culturing, and collecting CD3-cells;

3) ligating a 41BBL-MICA fusion gene to the lentiviral expression vector, followed by transferring into the competent cells to obtain a lentivirus containing the 41BBL-MICA fusion gene; and

4) mixing the CD3-cells obtained in step 2) with the lentivirus containing the 41BBL-MICA fusion gene obtained in step 3) and culturing to obtain the trophoblasts with limited generations; wherein

there is no time sequence limit between steps 1) and 3).

In some embodiments, a nucleotide sequence of the TAX2 gene in step 1) is set forth in SEQ ID No: 1.

In some embodiments, a nucleotide sequence of the 41BBL-MICA fusion gene in step 3) is set forth in SEQ ID No: 2.

In some embodiments, in steps 2) and 4), the culture time may be independently for not less than 14 d.

In some embodiments, in steps 2) and 4), the culture medium may be independently of a 1640 medium as a basic medium, including a fetal bovine serum with a mass percentage of 8% to 12% and interleukin-2 (IL-2) with a concentration of 180 IU/mL to 220 IU/mL.

The present disclosure further provides a culture method of SNK cells, including the following steps: obtaining trophoblasts by the preparation method, mixing the trophoblasts with PBMCs, followed by culturing for 14 d to 28 d to obtain SNK cells.

In some embodiments, a ratio of the number of trophoblasts to PBMCs may be (1-500):(1-10).

In some embodiments, the culture may be conducted at 35° C. to 42° C. with a CO2 volume concentration of 5%.

The present disclosure further provides a method for treating a tumor with SNK cells obtained by the culture method, wherein the tumor includes one or more selected from the group consisting of lung cancer, gastric cancer, colorectal cancer, liver cancer, glioma and breast cancer.

In some embodiments, when a tumor volume reaches 200 mm³, a first SNK cell reinfusion may be performed at a dosage of 5×10⁷ SNK cells; the reinfusion may be performed once a week.

The present disclosure provides a preparation method of trophoblasts with limited generations, including the following steps: 1) ligating a TAX2 gene to a lentiviral expression vector, followed by transferring into competent cells to obtain a lentivirus containing the TAX2 gene; 2) infecting PBMCs with the lentivirus containing the TAX2 gene obtained in step 1) and culturing, and collecting the CD3-cells; 3) ligating a 41BBL-MICA fusion gene to the lentiviral expression vector, followed by transferring into the competent cells to obtain a lentivirus containing the 41BBL-MICA fusion gene; and 4) mixing the CD3-cells obtained in step 2) with the lentivirus containing the 41BBL-MICA fusion gene obtained in step 3) and culturing to obtain the trophoblasts; wherein there is no time sequence limit between steps 1) and 3). The trophoblasts obtained by the preparation method provided by the present disclosure are of an autologous origin, so that the safety is ensured, irradiation is not required, and the operation is convenient.

The present disclosure further provides a culture method of SNK cells, including the following steps: obtaining trophoblasts by the preparation method described above, mixing the trophoblasts with PBMCs, followed by culturing for 14 d to 28 d to obtain SNK cells. The obtained trophoblasts can stimulate the expansion of CD56+CD16+NK cells in the PBMCs, and the obtained NK cells have a relatively strong killing effect on the various solid tumors.

The present disclosure further provides a method for treating a tumor with the SNK cells obtained by the culture method, wherein the SNK cells have therapeutic effects on lung cancer, gastric cancer, colorectal cancer, liver cancer, glioma and breast cancer.

### BRIEF DESCRIPTION OF THE DRAWINGS

FIG. 1 shows expression of MICA detected by flow cytometry;

FIG. 2 shows expression of 41BBL detected by flow cytometry;

FIG. 3 shows expression of CD3 detected by flow cytometry;

FIG. 4 shows the expansion of trophoblasts;

FIG. 5 shows a cell phenotype of SNK by flow cytometry;

FIG. 6 shows killing efficiencies of the SNK on different tumor cell lines;

FIG. 7 shows changes of a tumor volume in different tumor models;

FIG. 8 shows a CD56+CD16+ ratio in NK/SNK cells induced by different types of trophoblast stimulation;

FIG. 9 shows a growth curve of the NK/SNK cells induced by different types of trophoblast stimulation; and

FIG. 10 shows killing efficiencies of the NK/SNK cells induced by different types of trophoblast stimulation on various solid tumors.

### DETAILED DESCRIPTION OF THE EMBODIMENTS

The present disclosure provides a preparation method of trophoblasts with limited generations, including the following steps:

1) ligating a TAX2 gene to a lentiviral expression vector, followed by transferring into competent cells to obtain a lentivirus containing the TAX2 gene;

2) infecting peripheral blood mononuclear cells (PBMCs) with the lentivirus containing the TAX2 gene obtained in step 1) and culturing, and collecting CD3-cells;

3) ligating a 41BBL-MICA fusion gene to the lentiviral expression vector, followed by transferring into the competent cells to obtain a lentivirus containing the 41BBL-MICA fusion gene; and

4) mixing the CD3-cells obtained in step 2) with the lentivirus containing the 41BBL-MICA fusion gene obtained in step 3) and culturing to obtain the trophoblasts with limited generations; wherein

there is no time sequence limit between steps 1) and 3).

In the present disclosure, the TAX2 gene is ligated to the lentiviral expression vector and transferred into competent cells to obtain lentivirus containing the TAX2 gene.

The TAX2 gene is used to construct the trophoblasts with limited expansion generations (non-immortal); a nucleotide sequence of the TAX2 gene is set forth in SEQ ID No: 1, specifically as follows:

gcccatttcccaggatttggacagagcctcctatatggataccccgtct
acgtgtttggcgattgtgtacaggccgattggtgtcccgtctcaggtgg
tctatgttccacccgcctacatcgacatgccctcctggccacctgtcca
gagcaccaactcacctgggaccccatcgatggacgcgttgtcagctctc
ctctccaataccttatccctcgcctcccctccttccccacccagagaac
ctcaaggaccctcaaggtccttacccctcccaccactcctgtctccccc
aaggttccacctgccttctttcaatcaatgcgaaagcacaccccctacc
gaaatggatgcctggaaccaaccctcggggatcagctcccctccctcgc
cttccccgaacctggcctccgtccccaaaacatctacaccacctgggga
aaaaccgtagtatgcctatacctataccagctttccccacccatgacat
ggccacttataccccatgtcatattctgccaccccagacaattaggagc
cttcctcaccaaggtgcctctaaaacgattagaagaacttctatacaaa
atgttcctacacacagggacagtcatagtcctcccggaggacgacctac
ccaccacaatgttccaacccgtgagggct.

There is no particular limitation on the method for ligating the TAX2 gene to the lentiviral expression vector, and conventional genes can be used to ligate to the lentiviral expression vector. In some embodiments, the lentiviral expression vector includes a pCDH vector. There is no particular limitation on the method for transferring the lentiviral expression vector ligated with genes into the competent cells, and a conventional method will do. In some embodiments, the competent cells include Escherichia coli competent cells.

In the present disclosure, the PBMCs are infected with the lentivirus containing the TAX2 gene, and then are cultured, and CD3-cells are collected. In some embodiments, the culture time is not less than 14 d. In some embodiments, the culture medium is a 1640 medium as a basic medium, including a fetal bovine serum with a mass percentage of 8% to 12% and IL-2 with a concentration of 180 IU/mL to 220 IU/mL, in another embodiments, including a fetal bovine serum with a mass percentage of 10% and IL-2 with a concentration of 200 IU/mL. There is no particular limitation on the source of the above reagents, and a conventional commercial product will do. In some embodiments, the cultured cells are sorted using CD3 magnetic beads to collect the CD3-cells; there is no particular limitation on the method for sorting the CD3-cells using the CD3 magnetic beads, and a conventional method will do.

In the present disclosure, the 41BBL-MICA fusion gene is ligated to the lentiviral expression vector, followed by transferring into the competent cells to obtain lentivirus containing the 41BBL-MICA fusion gene. In the present disclosure, the 41BBL-MICA fusion gene has an effect of activating NK and stimulating NK expansion; a nucleotide sequence of the 41BBL-MICA fusion gene is set forth in SEQ ID No: 2, specifically as follows:

atggaatacgcctctgacgcttcactggaccccgaagccccgtggcctc
ccgcgccccgcgctcgcgcctgccgcgtactgccttgggccctggtcgc
ggggctgctgctgctgctgctgctcgctgccgcctgcgccgtcttcctc
gcctgcccctgggccgtgtccggggctcgcgcctcgcccggctccgcgg
ccagcccgagactccgcgagggtcccgagctttcgcccgacgatcccgc
cggcctcttggacctgcggcagggcatgtttgcgcagctggtggcccaa
aatgttctgctgatcgatgggcccctgagctggtacagtgacccaggcc
tggcaggcgtgtccctgacggggggcctgagctacaaagaggacacgaa
ggagctggtggtggccaaggctggagtctactatgtcttctttcaacta
gagctgcggcgcgtggtggccggcgagggctcaggctccgtttcacttg
cgctgcacctgcagccactgcgctctgctgctggggccgccgccctggc
tttgaccgtggacctgccacccgcctcctccgaggctcggaactcggcc
ttcggtttccagggccgcttgctgcacctgagtgccggccagcgcctgg
gcgtccatcttcacactgaggccagggcacgccatgcctggcagcttac
ccagggcgccacagtcttgggactcttccgggtgacccccgaaatccca
gccggactcccttcaccgaggtcggaagccacgaacttctctctgttaa
agcaagcaggagatgttgaagaaaaccccgggcctatggggctgggccc
ggtcttcctgcttctggctggcatcttcccttttgcacctccgggagct
gctgctgagccccacagtcttcgttataacctcacggtgctgtcctggg
atggatctgtgcagtcagggtttcttgctgaggtacatctggatggtca
gcccttcctgcgctatgacaggcagaaatgcagggcaaagccccaggga
cagtgggcagaagatgtcctgggaaataagacatgggacagagagacca
gggacttgacagggaacggaaaggacctcaggatgaccctggctcatat
caaggaccagaaagaaggcttgcattccctccaggagattagggtctgt
gagatccatgaagacaacagcaccaggagctcccagcatttctactacg
atggggagctcttcctctcccaaaacctggagactgaggaatggacagt
gccccagtcctccagagctcagaccttggccatgaacgtcaggaatttc
ttgaaggaagatgccatgaagaccaagacacactatcacgctatgcatg
cagactgcctgcaggaactacggcgatatctagaatccggcgtagtcct
gaggagaacagtgccccccatggtgaatgtcacccgcagcgaggcctca
gagggcaacatcaccgtgacatgcagggcttccagcttctatccccgga
atatcatactgacctggcgtcaggatggggtatctttgagccacgacac
ccagcagtggggggatgtcctgcctgatgggaatggaacctaccagacc
tgggtggccaccaggatttgccgaggagaggagcagaggttcacctgct
acatggaacacagcgggaatcacagcactcaccctgtgccctctgggaa
agtgctggtgcttcagagtcattggcagacattccatgtttctgctgtt
gctgctggctgctgctatttttgttattattattttctatgtccgttgt
tgtaa.

In the present disclosure, the 41BBL gene activates NK and stimulates NK expansion, and has a nucleotide sequence of the 41BBL gene is set forth in SEQ ID No: 3, specifically as follows:

atggaatacgcctctgacgcttcactggaccccgaagccccgtggcctc
ccgcgccccgcgctcgcgcctgccgcgtactgccttgggccctggtcgc
ggggctgctgctgctgctgctgctcgctgccgcctgcgccgtcttcctc
gcctgcccctgggccgtgtccggggctcgcgcctcgcccggctccgcgg
ccagcccgagactccgcgagggtcccgagctttcgcccgacgatcccgc
cggcctcttggacctgcggcagggcatgtttgcgcagctggtggcccaa
aatgttctgctgatcgatgggcccctgagctggtacagtgacccaggcc
tggcaggcgtgtccctgacggggggcctgagctacaaagaggacacgaa
ggagctggtggtggccaaggctggagtctactatgtcttctttcaacta
gagctgcggcgcgtggtggccggcgagggctcaggctccgtttcacttg
cgctgcacctgcagccactgcgctctgctgctggggccgccgccctggc
tttgaccgtggacctgccacccgcctcctccgaggctcggaactcggcc
ttcggtttccagggccgcttgctgcacctgagtgccggccagcgcctgg
gcgtccatcttcacactgaggccagggcacgccatgcctggcagcttac
ccagggcgccacagtcttgggactcttccgggtgacccccgaaatccca
gccggactcccttcaccgaggtcggaa.

In the present disclosure, a gene with a sequence of gccacgaacttctctctgtt aaagcaagcaggagatgttgaagaaaaccccgggcct is a linker gene.

In the present disclosure the MICA gene activates NK and stimulates NK expansion, and a nucleotide sequence of the MICA gene is set forth in SEQ ID No: 4, specifically as follows:

atggggctgggcccggtcttcctgcttctggctggcatcttcccttttg
cacctccgggagctgctgctgagccccacagtcttcgttataacctcac
ggtgctgtcctgggatggatctgtgcagtcagggtttcttgctgaggta
catctggatggtcagcccttcctgcgctatgacaggcagaaatgcaggg
caaagccccagggacagtgggcagaagatgtcctgggaaataagacatg
ggacagagagaccagggacttgacagggaacggaaaggacctcaggatg
accctggctcatatcaaggaccagaaagaaggcttgcattccctccagg
agattagggtctgtgagatccatgaagacaacagcaccaggagctccca
gcatttctactacgatggggagctcttcctctcccaaaacctggagact
gaggaatggacagtgccccagtcctccagagctcagaccttggccatga
acgtcaggaatttcttgaaggaagatgccatgaagaccaagacacacta
tcacgctatgcatgcagactgcctgcaggaactacggcgatatctagaa
tccggcgtagtcctgaggagaacagtgccccccatggtgaatgtcaccc
gcagcgaggcctcagagggcaacatcaccgtgacatgcagggcttccag
cttctatccccggaatatcatactgacctggcgtcaggatggggtatct
ttgagccacgacacccagcagtggggggatgtcctgcctgatgggaatg
gaacctaccagacctgggtggccaccaggatttgccgaggagaggagca
gaggttcacctgctacatggaacacagcgggaatcacagcactcaccct
gtgccctctgggaaagtgctggtgcttcagagtcattggcagacattcc
atgtttctgctgttgctgctggctgctgctatttttgttattattattt
tctatgtccgttgttgtaa.

There is no particular limitation on the method for ligating the 41BBL-MICA fusion gene to the lentiviral expression vector, and conventional genes can be used to ligate to the lentiviral expression vector. In some embodiments, the lentiviral expression vector includes a pCDH vector. There is no particular limitation on the method for transferring the lentiviral expression vector ligated with genes into the competent cells, and a conventional method will do. In some embodiments, the competent cells include Escherichia coli competent cells.

In the present disclosure, the CD3-cells are mixed with the lentivirus containing the 41BBL-MICA fusion gene, and then are cultured to obtain the trophoblasts. In some embodiments, the culture time is not less than 14 d;. In some embodiments, the culture medium is a 1640 medium as a basic medium, including a fetal bovine serum with a mass percentage of 8% to 12% and IL-2 with a concentration of 180 IU/mL to 220 IU/mL, in another embodiment, including a fetal bovine serum with a mass percentage of 10% and IL-2 with a concentration of 200 IU/mL. There is no particular limitation on the source of the above reagents, and a conventional commercial product will do. In some embodiments, the cultured cells are sorted using a flow sorter, and the obtained CD3−41BBL+MICA+ are the trophoblasts; there is no particular limitation on the sorting method using the flow sorter, and a conventional method will do.

The present disclosure further provides a culture method of SNK cells, including the following steps: obtaining the trophoblasts by the preparation method, mixing the trophoblasts with the PBMCs, followed by culturing for 14 d to 28 d to obtain SNK cells.

In some embodiments, a ratio of the number of trophoblasts to PBMCs is (1-500):(1-10), and in another embodiment, it is 200:1. In some embodiments, a temperature of culture is in a range of 35° C. to 42° C., in another embodiment, it is 37° C. A volume concentration of CO₂ in the culture is 5%.

In some embodiments, the culture medium is a 1640 medium as a basic medium, including a fetal bovine serum with a mass percentage of 8% to 12% and IL-2 with a concentration of 180 IU/mL to 220 IU/mL, in another embodiments, including a fetal bovine serum with a mass percentage of 10% and IL-2 with a concentration of 200 IU/mL. In some embodiments, 50 IU/mL to 500 IU/mL of the IL-2 is supplemented every day during the culture; in some embodiments, when the medium becomes yellow during the culture, the medium is changed at a half dosage.

In the present disclosure, the SNK cells are Super-NK cells, referred to as SNK cells, which have relatively strong killing effects on various solid tumors. In some embodiments, the solid tumors include lung cancer, gastric cancer, breast cancer, liver cancer, colorectal cancer and glioma.

The present disclosure further provides a method for treating a tumor with SNK cells obtained by the culture method, wherein the tumors include one or more selected from the group consisting of lung cancer, gastric cancer, colorectal cancer, liver cancer, glioma and breast cancer. In the present disclosure, when the tumor volume reaches 200 mm³, a first SNK cell reinfusion is performed at a dosage of 5×10⁷ SNK cells; the reinfusion is performed once a week for a total of 6 times.

The technical schemes provided by the present disclosure will be described in detail below with reference to examples, but they should not be construed as limiting the protection scope of the present disclosure.

### EXAMPLE 1

Culture of trophoblasts with limited expansion generations

1. Cells with limited expansion generations

Gene synthesis: TAX2 gene (SEQ ID No: 1) and 41BBL-MICA fusion gene (SEQ ID No: 2) were synthesized by GENEWIZ, Inc.; the genes were ligated to a lentiviral expression vector pCDH, and a viral supernatant was harvested by lentiviral packaging, followed by concentration and transfection into PBMCs; the specific operation process were as follows:

the lentivirus was transferred into E. coli competent cells, positive clones were screened, and the screened positive clones were sequenced and the correct ones were stored for later use;

a) 293T cells with a cell density of about 3×10⁶ cells/10 ml/T75 were inoculated into 3 bottles, the mixture was shaken well and cultured at 37° C. in a CO₂ incubator overnight; on the next day, lentivirus was packaged when the cell density reached 80%;

b) 1.5 ml of opti-MEM was added to two 15 ml centrifuge tubes A and B, respectively; 60 μg of plasmid was added to the tube A, and mixed well, 300 ng of Liposome 3000 was added to the tube B, and mixed well; stood at room temperature for 5 min;

c) after 5 min, the liquid in the tube B was added to the tube A, and mixed well (with a force lighter than the first time), followed by standing at room temperature for 30 min;

d) a supernatant was discarded from the 293T cells with a density of 80%, 5 ml of the opti-MEM was added to wash once, followed by adding 4 ml of the opti-MEM;

e) after 30 min, a mixture of A and B was gently added dropwise into the 293T cells with an average of 1 ml per bottle, and shaken gently, followed by incubation in a CO₂ incubator at 37° C. for 4 h;

f) after the incubation, 10 ml of a fresh opti-MEM medium was replaced, followed by shaking gently and conducting incubation in a CO₂ incubator at 37° C. overnight;

g) after 48 h of incubation, a viral supernatant was harvested and stored in a refrigerator at 4° C., and the 293T cells were further cultured after adding 10 ml of an opti-MEM medium; and

h) after culture for 72 h, a viral supernatant was harvested and combined with the viral supernatant harvested at 48 h, the combined supernatant was centrifugated at 2,000 rpm at 4° C. for 10 min; the obtained supernatant was filtered through a 0.45 μm syringe filter, and concentrated by centrifugation on a virus concentration column (60 ml of viral supernatant), followed by centrifugation at 3,000 rpm for 30 min; the obtained substance was resuspended in 300 μl of sterile PBS, and the obtained viral supernatant was freeze-stored at −80° C. for later use.

3. Isolation of PBMCs:

a) 50 ml of peripheral blood samples were collected from three healthy volunteers with anticoagulant blood collection tubes, named A, B and C, respectively;

b) the three peripheral blood samples were transferred into 250 ml centrifuge tubes, an equal volume of PBS (50 ml) was added into each tube, followed by gently pipetting to form a cell suspension;

c) another 50 ml centrifuge tubes were added with 20 ml of a lymphocyte separation solution (GE), the cell suspension obtained in step b) was aspirated with a pipette in an amount such that the ratio of the cell separation solution to the cell suspension was 1:1, and the cell suspension was added carefully and slowly above the centrifuge tube to overlap the cell suspension on the lymphocyte separation solution, followed by centrifugation at 2,000 rpm for 20 min;

d) the centrifuge tube was taken out, an uppermost layer of plasma was removed using a pipette, and mononuclear cells in a middle buffy coat layer was sucked up using a pipette and put into a new centrifuge tube; 10 ml of an RPMI-1640 medium was added, followed by gently pipetting evenly and then centrifugation at 2,000 rpm for 5 min; the resulted supernatant was removed, followed by washing 2 times in total; and

e) the obtained peripheral blood mononuclear cells were counted after the last washing, the cell density was adjusted to 1×10⁶ cells/ml and the cells were inoculated into a 6-well cell culture plate at 8 ml/well, each group of PBMCs were inoculated into 3 wells, where the cell culture medium was RPMI-1640+10% FBS, and the cells were labeled as D0.

4. Culture of trophoblasts with limited expansion generations:

a) lentivirus infection of PBMCs: a frozen 300 μl TAX2 lentivirus concentrate was thawed and added to isolated 1×10⁶ cell PBMCs, followed by culture with 1640+10% FBS+200 IU/mL IL-2 for 14 d;

b) CD3-cells were sorted with CD3 magnetic beads and collected, at this time the cells were cells with limited expansion generations; and

c) 1×10⁶ cells of limited expansion generations were added with 300 μl of a 41BBL-MICA fusion gene-containing lentivirus concentrate after thawing, the obtained mixture was cultured with 1640+10% FBS+200 IU/mL IL-2 for 14 d;

CD3−41BBL+MICA+ cells were separated by a flow sorter; obtained cells were trophoblasts with limited expansion generations, and the obtained cells were cultured with 1640+10% FBS+200 IU/mL IL-2.

The obtained trophoblasts of limited expansion generations were identified by HLA typing, and HLA sequencing was conducted by Beijing GenomePrecision Technology Co., Ltd. The results were as follows: HLA-A1101, HLA-A2402, HLA-B1511, HLA-B1505, HLA-00303 and HLA-00401.

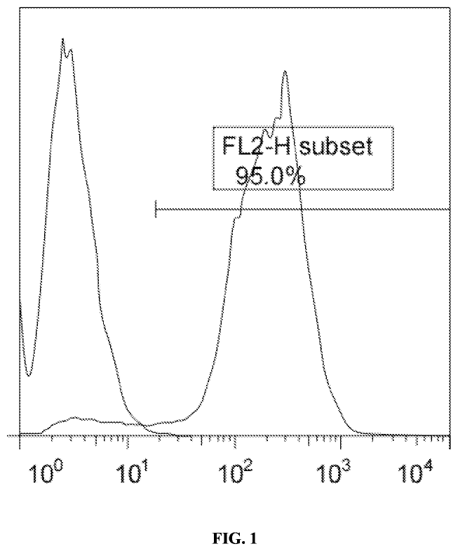

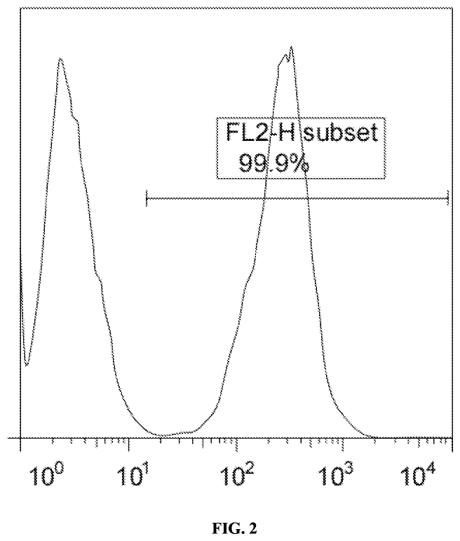

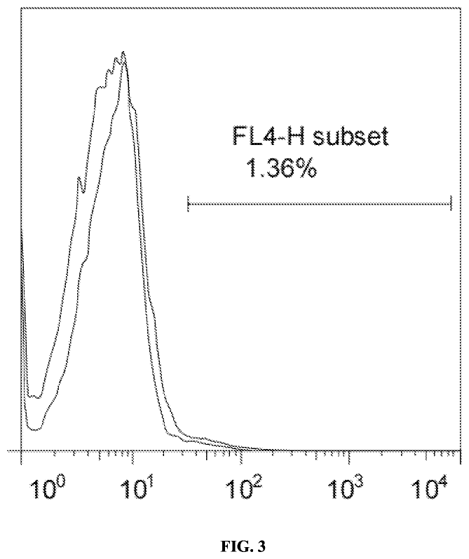

Flow sorting of CD3−41BBL+MICA+ cells: the CD3−41BBL+MICA+ cells were sorted with a Sony flow cytometer; the sorted cells were subjected to flow cytometry, and the expressions of CD3, 41BBL and MICA were shown in FIG. 1 to FIG. 3, and the trophoblasts were CD3−MICA+41BBL+.

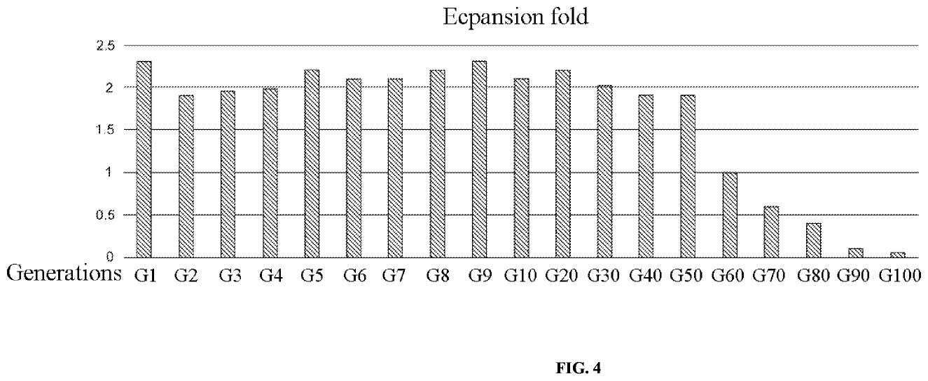

Expansion of trophoblasts with limited expansion generations: during the culture of trophoblasts, the medium was changed and centrifuged every other day, this was marked as 1st generation; the number of cells in the next generation/the number of cells in the previous generation was regarded as an expansion fold; the expansion of the obtained trophoblasts was accounted, and the results were shown in FIG. 4. Before a 50th generation, the cells basically maintained a 2-fold expansion; after the 50th generation, the expansion capacity gradually decreased; in the 60th generation, there was basically no expansion; after the 60th generation, the trophoblasts died one after another, and the number of cells gradually decreased. Therefore, trophoblasts had the limited expansion generations.

### EXAMPLE 2

1. Culture of SNK cells:

a) human PBMCs were isolated, centrifuged and counted, followed by resuspending with 1640+10%FBS+200 IU/mL IL-2;

b) the trophoblasts of limited expansion generations obtained in Example 1 were added in a ratio of 200:1; the obtained mixture was incubated at 37° C. in a CO₂ incubator;

c) according to an actual volume, 50 IU/mL to 500 IU/mL of IL-2 was supplemented every day; and according to a growth of the cells, when the medium turned yellow, the medium was changed preferably at a half dosage; and

d) SNK cells were obtained when cultured for 14 d to 28 d.

2. Detection of SNK phenotype by flow cytometry

The cells were cultured for 14 d, and the expressions of CD3, CD4, CD8, CD56 and CD16 were detected by flow cytometry, and the SNK phenotype was analyzed. Specific steps were as follows:

a) SNK cells cultured for 14 d were centrifuged at 1,000 rpm for 5 min, the supernatant was discarded, the resulted substance was washed with 10 ml of the PBS, and the washing solution was discarded;

b) the cells were resuspended with 6 ml of the PBS, and divided into 5 flow tubes, with 1 ml per tube, the cell suspension after resuspension was centrifugated at 1,000 rpm for 5 min, the supernatant was discarded, and the cells in each tube were resuspended with 50 μl of the PBS;

c) incubation of antibodies: there were 6 tubes for each group of cells, respectively, including: a negative control group without antibodies; single staining tubes was added with 10 μl of CD3, CD4, CD8, CD56 and CD16, respectively, as sample tubes; the cells were incubated at room temperature for 30 min in the dark; and

d) after antibody incubation, 2 ml of the PBS was added to each tube to wash, the obtained mixture was centrifugated at 1,000 rpm for 5 min, the supernatant was discarded, and 1 ml of the PBS was added to resuspend, the obtained resuspended cell suspension was loaded on a flow cytometer for detection.

3. Co-culture of NK and target cells to detect killing efficiencies

When the cells were cultured for 14 d, the killing efficiencies of lung cancer cells NCI-H358, colorectal cancer cells SW48, glioma cells U87, liver cancer cells HepG2, breast cancer cells MCF7 and gastric cancer NCI-N87 cells were detected, respectively; the specific steps were as follows:

a) different target cells were gently scraped with a cell scraper, the target cells were washed with 10 ml of PBS and collected in a centrifuge tube, the resulted mixture was centrifugated at 1,000 rpm for 5 min, the supernatant was discarded; the cells were resuspended in 10 ml of RPMI-1640+2% FBS, the cells was adjusted to a cell density of 8×10⁴ cells/ml according to the number of cells after sampling and counting;

b) Inoculation of target cells: the target cells after adjusting the density were inoculated into a 96-well cell culture plate with a multi-channel pipetting gun, with 50 μl per well, and a control group was set without target cells, and 50 μl of RPMI-1640+2%FBS was added to each well;

c) SNK cells cultured for 14 d were centrifuged, resuspended in 30 ml of PBS, the suspension was sampled and counted, followed by centrifugation and washing, and the supernatant was discarded; the cells were resuspended with the RPMI-1640+2% FBS according to counting results, and the cell density was adjusted to 3.2×10⁶ cells/ml, the adjusted suspension was serially diluted to 1.6×10⁶ cells/ml, 0.8×10⁶ cells/ml, 0.4×10⁶ cells/ml, 0.2×10⁶ cells/ml, 0.1×10⁶ cells/ml;

d) inoculation of SNK: the diluted SNK of different densities were added to the corresponding 96-well plates containing target cells in sequence, with 50 μl/well, such that effector-target ratio were 40:1, 20:1, 10:1, 5:1, 2.5:1 and 1.25:1; T-high and T-low groups were set, and added with 50 μl of RPMI-1640+2% FBS;

e) after inoculation, the cells were co-cultured at 37° C. in a CO₂ incubator for 4 h;

f) 10 μl of a cell lysate was added to each well of the T-high group after the cells co-culture for 4 h, and the cells were fully lysed in a CO₂ incubator at 37° C. for 1 h;

g) after the lysis, the cells were taken out, and 100 μl of a Working Solution was added to each well, followed by protecting from light at room temperature for 30 min; and

h) after adding 50 μl of a Stop Solution to each well, an absorbance was immediately measured at 490 nm with a microplate reader, and the cell killing efficiency was calculated according to the formula.

4. In vivo efficacy experiment of SNK-immunodeficiency mouse model

Mouse tumor-bearing models were established with lung cancer H358, gastric cancer N87, colorectal cancer SW48, liver cancer HepG2, glioma U87 and breast cancer MCF7, respectively.

5-week-old female mice were selected, with 6 mice in each group; when the tumor grew to 200 mm³ to 300 mm³, 5×10⁷ SNK cells were reinfused at a dosing interval of once a week, where the 5×10⁷ SNK cells were reinfused each time, and the tumor was measured before the reinfusion.

The results were as follows:

SNK cell phenotype detection by flow cytometry:

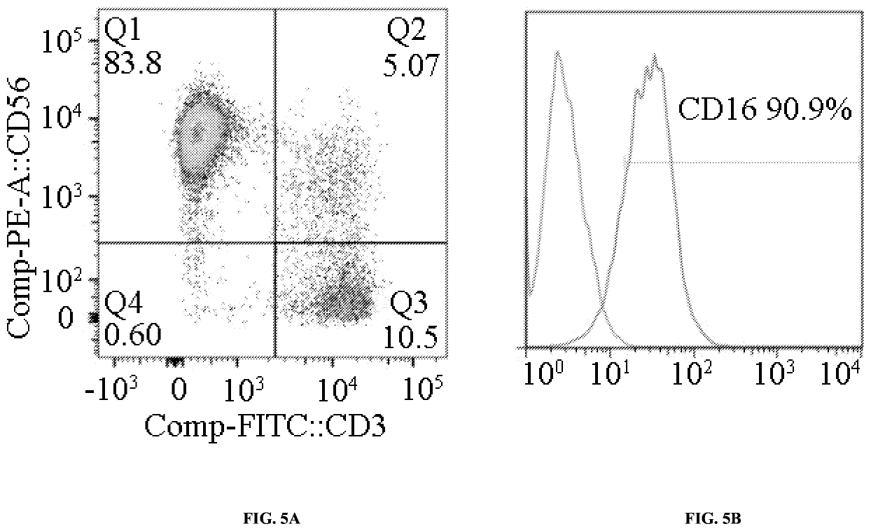

The SNK cells were detected by flow cytometry after culturing for 14 d, and the results are shown in FIG. 5. The percentage of CD56+CD3− in NK cells was 83.8%, the percentage of CD56+CD3+ in NKT cells was 5.07%, and among the CD56+CD3− NK cells, 90.9% of the cells were CD16+.

The killing effect of SNK cells on various solid tumors:

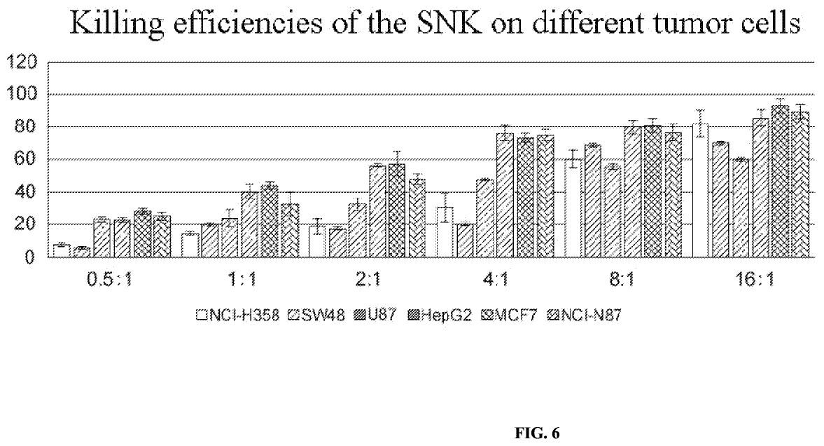

The killing efficiency of SNK cells against different tumor cells was detected by lactate dehydrogenase (LDH) method: lung cancer H358, gastric cancer N87, colorectal cancer SW48, liver cancer HepG2, glioma U87 and breast cancer MCF7, and the results were shown in FIG. 6. The killing efficiency of the SNK on different tumor cells increased with an increase of the effector-target ratio, and at 16:1, the killing efficiency exceeded 60%.

In vivo efficacy experiment of SNK:

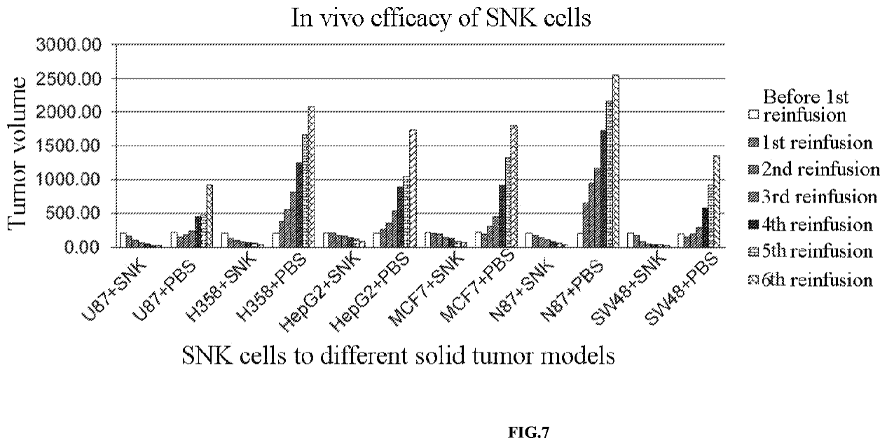

Mouse tumor-bearing models were established with the lung cancer H358, gastric cancer N87, colorectal cancer SW48, liver cancer HepG2, glioma U87 and breast cancer MCF7, respectively; when the tumor volume grew to 200 mm³, the first SNK cell reinfusion is conducted at a dosage of 5×10⁷ SNK cells per mouse, where the reinfusion is conducted once a week for a total of 6 times; the mice of control group are reinfused with the PBS. The tumor volume was shown in FIG. 7, the changes of the tumor volume with different cell lines were slightly different; the tumor volume of mice reinfused with PBS was increased to different degrees, while the tumor volume of mice reinfused with SNK cells showed a downward trend, indicating that the SNK cells had significant tumor clearance effects on the lung cancer, gastric cancer, breast cancer, liver cancer, colorectal cancer and glioma.

### EXAMPLE 3

K562 cells were infected with the lentivirus concentrate of the 41BBL-MICA fusion gene in Example 1, and K562 cells expressing 41BBL and MICA were obtained by flow sorting; the K562 cells expressing 41BBL and MICA were used as trophoblasts, and induction culture of NK was conducted by the culture method in Example 2. The NK cells were compared with SNK induced by trophoblasts with limited generations in the present disclosure.

CD56+CD16+ ratio in NK cells induced by different types of trophoblasts

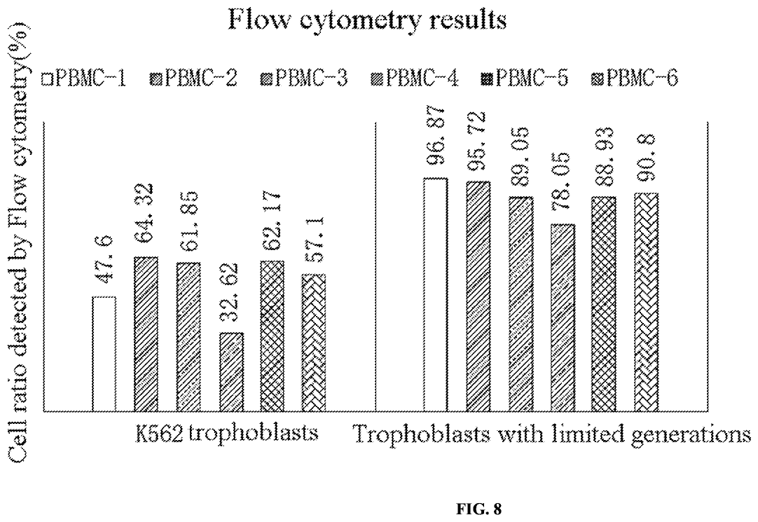

6 PBMCs from different sources were subjected to stimulation and induction of NK/SNK in different trophoblast systems, and the results were shown in FIG. 8. In the NK cells induce-cultured by K562 trophoblasts, the NK ratio of CD56+CD16+ was 32.62% to 64.32%; while in the SNK induce-cultured by the trophoblasts with limited generations, the NK ratio of CD56+CD16+ was 78.05% to 96.87%. In the PBMCs from a same source, the NK ratio of CD56+CD16+ induced by trophoblasts with limited generations was higher. These results indicated that the type of trophoblast is the key factor affecting its different cell ratio.

Growth curves of cell expansion induced by different trophoblasts

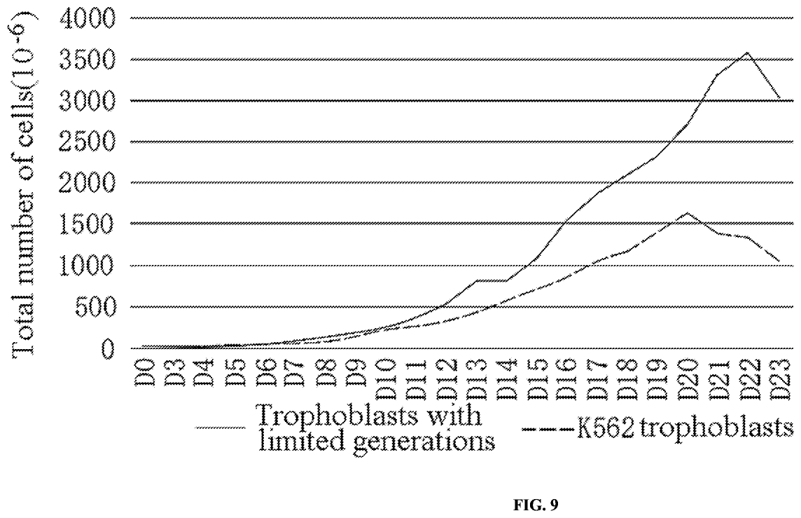

The growth curve of NK/SNK induced by different trophoblast stimulations was measured with PBMC-1, and the results were shown in FIG. 9. The SNK cells stimulated by the trophoblasts with limited generations expand faster and at higher folds, and the number of cells obtained in final culture was 3 times that of K562-induced NK cells.

Killing efficiencies of the NK/SNK cells induced by different types of trophoblasts on various solid tumors

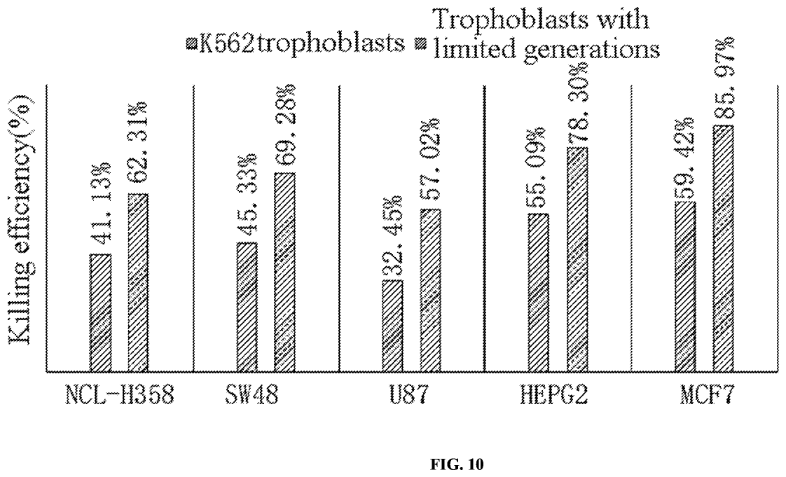

The killing efficiencies of the NK/SNK induced by stimulation with PBMC-6 source on lung cancer H358, colorectal cancer SW48, liver cancer HepG2, glioma U87 and breast cancer MCF7 were detected and compared by LDH method. The results were shown in FIG. 10, the killing efficiency of SNK stimulated by the trophoblasts with limited generations on various solid tumor cell lines is significantly higher than that of the K562-based trophoblasts. This shows that the type of trophoblast is the key factor affecting its function.

The above descriptions are only descriptions of the preferred embodiments of the present disclosure. It should be noted that, for those skilled in the art, several improvements and modifications will be made, without departing from the principle of the present disclosure, but such improvements and modifications should be deemed as falling within the protection scope of the present disclosure.

1. A preparation method of trophoblasts with limited generations, comprising the following steps:
1) ligating a TAX2 gene to a lentiviral expression vector, followed by transferring into competent cells to obtain a lentivirus containing the TAX2 gene;
2) infecting peripheral blood mononuclear cells (PBMCs) with the lentivirus containing the TAX2 gene obtained in step 1) and culturing, and collecting CD3-cells;
3) ligating a 41BBL-MICA fusion gene to the lentiviral expression vector, followed by transferring into the competent cells to obtain a lentivirus containing the 41BBL-MICA fusion gene; and
4) mixing the CD3-cells obtained in step 2) with the lentivirus containing the 41BBL-MICA fusion gene obtained in step 3) and culturing to obtain the trophoblasts with limited generations; wherein
there is no time sequence limit between steps 1) and 3).

2. The preparation method according to claim 1, wherein a nucleotide sequence of the TAX2 gene in step 1) is set forth in SEQ ID No: 1.

3. The preparation method according to claim 1, wherein a nucleotide sequence of the 41BBL-MICA fusion gene in step 3) is set forth in SEQ ID No: 2.

4. The preparation method according to claim 1, wherein a culture time is independently for not less than 14 d in steps 2) and 4).

5. The preparation method according to claim 1, wherein in steps 2) and 4), a culture medium is independently of a 1640 medium as a basic medium, comprising a fetal bovine serum with a mass percentage of 8% to 12% and interleukin-2 (IL-2) with a concentration of 180 IU/mL to 220 IU/mL.

6. A culture method of SNK cells, comprising the following steps: obtaining trophoblasts by the preparation method according to claim 1, mixing the trophoblasts with PBMCs, followed by culturing for 14 d to 28 d to obtain SNK cells.

7. The culture method according to claim 6, wherein a ratio of the number of trophoblasts to PBMCs is (1-500):(1-10).

8. The culture method according to claim 6, wherein the culture is conducted at 35° C. to 42° C. with a CO₂ volume concentration of 5%.

9. A method for treating a tumor with SNK cells obtained by the culture method according to claim 6, wherein the tumors comprise one or more selected from the group consisting of lung cancer, gastric cancer, colorectal cancer, liver cancer, glioma and breast cancer.

10. The method according to claim 9, wherein when a tumor volume reaches 200 mm³, a first SNK cell reinfusion is performed at a dosage of 5×10⁷ SNK cells; the reinfusion is performed once a week for a total of 6 times.

11. The preparation method according to claim 4, wherein in steps 2) and 4), the culture medium is independently of a 1640 medium as a basic medium, comprising a fetal bovine serum with a mass percentage of 8% to 12% and interleukin-2 (IL-2) with a concentration of 180 IU/mL to 220 IU/mL.

12. The culture method of SNK cells according to claim 6, wherein in step 1) of the preparation method for prepare the trophoblasts with limited generations, a nucleotide sequence of the TAX2 gene is set forth in SEQ ID No: 1.

13. The culture method of SNK cells according to claim 6, wherein in step 3) of the preparation method for prepare the trophoblasts with limited generations, a nucleotide sequence of the 41BBL-MICA fusion gene is set forth in SEQ ID No: 2.

14. The culture method of SNK cells according to claim 6, wherein in steps 2) and 4) of the preparation method for prepare the trophoblasts with limited generations, a culture time is independently for not less than 14 d.

15. The culture method of SNK cells according to claim 6, wherein in steps 2) and 4) of the preparation method for prepare the trophoblasts with limited generations, a culture medium is independently of a 1640 medium as a basic medium, comprising a fetal bovine serum with a mass percentage of 8% to 12% and interleukin-2 (IL-2) with a concentration of 180 IU/mL to 220 IU/mL.

16. The method according to claim 9, wherein a ratio of the number of trophoblasts to PBMCs is (1-500):(1-10).

17. The method according to claim 9, wherein the culture is conducted at 35° C. to 42° C. with a CO₂ volume concentration of 5%.
